(19) United States
(12) Patent Application Publication
(10) Pub. No.: US 2025/0389175 A1
(43) Pub. Date: Dec. 25, 2025

(54) HYDROGEN GENERATION WITHIN HIGH SALINITY HYDROCARBON-BEARING RESERVOIRS

(71) Applicant: Chevron U.S.A. Inc., San Ramon, CA (US)

(72) Inventors: Wei Shi, Houston, TX (US); Paul J. Evans, Katy, TX (US)

(21) Appl. No.: 19/237,746

(22) Filed: Jun. 13, 2025

Related U.S. Application Data
(60) Provisional application No. 63/662,327, filed on Jun. 20, 2024.

(51) Int. Cl. E21B 43/25; C09K 8/84

(52) U.S. Cl. CPC E21B 43/25; C09K 8/845

(57) ABSTRACT

A process includes identifying a hydrocarbon-bearing reservoir containing a hydrocarbon fluid and a brine having a sufficient salinity level to prevent or inhibit one or more of methanogenesis and sulphate-reduction activity, and responsive to a concentration of hydrogen being below 1000 parts per million in a sample collected from the hydrocarbon-bearing reservoir, introducing at least one non-native or indigenous hydrogen producing microorganism or stimulating the activity of at least one indigenous bacteria capable of stimulating a microbial consortium to increase a production rate of hydrogen in the hydrocarbon-bearing reservoir.

### PRIORITY CLAIM

The present application claims priority to U.S. Provisional Patent Application Ser. No. 63/662,327, entitled “Hydrogen Generation within High Salinity Hydrocarbon-Bearing Reservoirs,” filed Jun. 20, 2024, the content of which is incorporated by reference herein in its entirety.

### BACKGROUND

Hydrogen provides the means of both the storage and transport of energy. As an energy carrier, it is a commodity like the other carriers (e.g., electricity, coal, natural gas, gasoline, etc.). Hydrogen can be produced from diverse sources, including fossil fuels, biomass, industrial waste, and electrolysis, and then can be consumed in any sector: transportation, electric, natural gas, or even utilized as a feedstock in many industries.

Hydrogen generation is a key component for achieving a sustainable, low-carbon future. Its applications span from clean transportation to industrial processes, making it a key component in the transition toward a greener economy. For example, some applications include distributed or combined-heat-and-power; backup power; systems for storing and enabling renewable energy; portable power; auxiliary power for trucks, aircraft, rail, and sea vessels; specialty vehicles such as forklifts; and passenger and freight vehicles including cars, trucks, and buses. For industrial applications, hydrogen has the potential to replace natural gas for heating and electricity generation by co-generation or by blending directly into the natural gas grid. Hydrogen can provide a feedstock in industries such as oil refineries, ammonia production, metallic ore reduction, hydrochloric acid production, hydrogenating agent, etc. Hydrogen can be transported in a gaseous or liquid state. Gaseous hydrogen can be transported physically through a number of means, including road, rail, sea, air, or pipeline transport. Hydrogen can also be transported on an energy basis through the natural gas or electrical grids.

### SUMMARY

In accordance with an illustrative embodiment, a process comprises:
identifying a hydrocarbon-bearing reservoir containing a hydrocarbon fluid and a brine with a sufficient salinity level to prevent or inhibit one or more of methanogenesis and sulphate-reduction activity, and
responsive to a concentration of hydrogen being below 1000 parts per million in a sample collected from the hydrocarbon-bearing reservoir, introducing at least one non-native or indigenous hydrogen producing microorganism or stimulating the activity of at least one indigenous bacteria capable of stimulating a microbial consortium to increase a production rate of hydrogen in the hydrocarbon-bearing reservoir.

In accordance with another illustrative embodiment, a process comprises:
identifying a hydrocarbon-bearing reservoir containing a hydrocarbon fluid and a brine with a sufficient salinity level to prevent or inhibit one or more of methanogenesis and sulphate-reduction activity, and
responsive to a concentration of hydrogen being equal to or greater than 1000 parts per million in a sample collected from the hydrocarbon-bearing reservoir, collecting at least a portion of a hydrogen-rich material comprising hydrogen from the hydrocarbon-bearing reservoir.

### DETAILED DESCRIPTION

Various illustrative embodiments described herein are directed to hydrogen generation within high salinity hydrocarbon-bearing reservoirs using anaerobic microbial fermentation. The generation of hydrogen into added value chemicals, materials and fuels offers one alternative to crude.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology can be applied, as long as that definition does not conflict with any other disclosure or definition applied herein or render indefinite or non-enabled any claim to which that definition is applied. To the extent that any definition or usage provided by any document incorporated herein by reference conflicts with the definition or usage provided herein, the definition or usage provided herein controls.

While systems and processes are described in terms of “comprising” various components or steps, the systems and processes can also “consist essentially of” or “consist of” the various components or steps, unless stated otherwise.

The terms “a,” “an,” and “the” are intended to include plural alternatives, e.g., at least one. The terms “including,” “with,” and “having,” as used herein, are defined as comprising (i.e., open language), unless specified otherwise.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. For example, all numerical end points of ranges disclosed herein are approximate, unless excluded by proviso.

Values or ranges may be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means ±20% of the stated value, ±15% of the stated value, +10% of the stated value, ±5% of the stated value, ±3% of the stated value, or ±1% of the stated value.

Applicant reserves the right to proviso out or exclude any individual members of any such group of values or ranges, including any sub-ranges or combinations of sub-ranges within the group, that can be claimed according to a range or in any similar manner, if for any reason Applicant chooses to claim less than the full measure of the disclosure, for example, to account for a reference that Applicant may be unaware of at the time of the filing of the application. Further, Applicant reserves the right to proviso out or exclude any members of a claimed group.

The term “primarily” shall be understood to mean an amount greater than 50%, e.g., 50.01 to 100%, or any range between, e.g., 51 to 95%, 75% to 90%, at least 60%, at least 70%, at least 80%, etc.

The term “non-native” as used herein refers to a microorganism that is not naturally occurring in a particular location, such as a particular subterranean formation.

The term “hydrocarbon fluid” as used herein refers to a hydrocarbon-bearing fluid, such as crude oil, natural gas, petroleum, diesel fuel, gasoline, or any other fluids that include an amount of hydrocarbons. Moreover, this term may include fluids of all phases, such as any substance that continually deforms (flows) under an applied shear stress, or external force. Examples of such substances include liquids, gases, and plasmas.

The term “production well” as used herein is defined as a well that enables oil or gas to be extracted from an oil-containing underground rock formation or a gas-containing underground rock formation.

The term “brine” as used herein refers to an aqueous solution of salts and other water-soluble compounds. The term “water” can be used interchangeably with the term “brine” herein.

The term “salinity” as used herein refers to a concentration of dissolved salts in an aqueous brine and is reported in this disclosure in units of parts per thousand (ppt).

As stated above, hydrogen generation is a key component for achieving a sustainable, low-carbon future. Hydrogen can be produced from diverse sources, including fossil fuels, biomass, industrial waste, and electrolysis, and then can be consumed in any sector: transportation, electric, natural gas, or even utilized as a feedstock in many industries.

There are many oil and gas fields within the United States and around the world that are at or near the point of abandonment due to the inability to continue to produce oil or gas from them profitably. Under current technology, those fields will be abandoned with billions of barrels of oil remaining in place since primary and secondary oil recovery techniques still normally leave behind half or more of the original oil in place in those reservoirs at the time they are abandoned. Accordingly, the remaining oil represents a significant quantity of material for the generation of hydrogen that would otherwise be lost.

Microorganisms are found throughout oil production systems, from the reservoir rock itself, through pipelines and topsides facilities. For example, sulphate-reducing bacteria are responsible for the majority of the bacterial problems in oil production. Hydrogen sulphide is produced directly by sulphate-reducing bacteria as a by-product of respiration. This hazardous gas, a respiratory inhibitor, is volatile and toxic. It also makes it more difficult to refine crude oil and gas into environmentally friendly, high-quality fuels, hence reducing its value.

One mechanism for hydrogen generation is enhanced anaerobic microbial fermentation of residual oil in the depleted oil and gas reservoirs. The common reaction products from anacrobic fermentation of hydrocarbon compounds include hydrogen, acetic acid and carbon dioxide. The fermentation process can be stimulated by the introduction of surfactants to increase emulsification and oil-water surface area, and the injection of bacterial nutrients to increase the activity of the mixed microbial community growing on hydrocarbons. This process is sometimes referred to as dark fermentation as it is taking place in the absence of light (photo-fermentation). The primary issue with this hydrogen generation mechanism is that the hydrogen generated is rapidly consumed as an electron donor in other types of microbial activity, such as sulphate reduction and methanogenesis thereby generating undesired products such as H₂S and CH₄.

In view of these challenges, there is a need for solutions that can generate hydrogen in existing hydrocarbon-bearing reservoirs while preventing or inhibiting the consumption of hydrogen as an electron donor in other types of microbial activity, such as sulphate reduction and methanogenesis. The non-limiting illustrative embodiments described herein advantageously extend the use of existing oil and gas reservoirs indefinitely by utilizing the infrastructure therein such as drilled and cased wells to access and manipulate those reservoirs to generate hydrogen production for beneficial use. This, in turn, reduces the need to drill new reservoirs in potentially environmentally sensitive areas to access more hydrocarbon fluids such as oil and natural gas. By utilizing the existing infrastructure, the capital expenditures can be significantly reduced while simultaneously generating a valuable energy source and minimizing the number of reservoirs to be drilled.

The non-limiting illustrative embodiments described herein overcome the drawbacks discussed above by providing processes for generating hydrogen by enhanced anaerobic microbial fermentation in a hydrocarbon-bearing reservoir containing a hydrocarbon fluid and a brine having a sufficient salinity level to prevent or inhibit one or more of methanogenesis and sulphate-reduction activity, i.e., prevent or inhibit the rapid consumption of the generated hydrogen as an electron donor in other types of microbial activity, such as sulphate reduction and methanogenesis (i.e., generating H₂S or CH₄). It has advantageously been discovered that anaerobic microbial fermentation can take place in a hydrocarbon-bearing reservoir at a high salinity level, while the activity of sulphate reducing bacteria and hydrogenotrophic methanogens is prevented or inhibited. In particular, anaerobic fermentation generates more energy than, for example, sulphate or carbon dioxide reduction, thereby allowing the fermentative microbes to manage osmotic pressure. For example, bacteria need water to survive, and high salinity brines mean that water will diffuse out of the cells due to osmosis.

In a non-limiting illustrative embodiment, a process includes at least identifying a hydrocarbon-bearing reservoir containing a hydrocarbon fluid and a brine having a sufficient salinity level to prevent or inhibit one or more of methanogenesis and sulphate-reduction activity (i.e., a salinity being in a methanogenesis and/or sulphate-reduction activity inhibiting amount). In some embodiments, a hydrocarbon-bearing reservoir can be a subsurface reservoir. In some embodiments, a subsurface reservoir can be a subterranean reservoir or a subsea reservoir. In some embodiments, a hydrocarbon-bearing reservoir can be a horizontal well or a vertical well. In some embodiments, a horizontal well or a vertical well of a hydrocarbon-bearing reservoir can include one or more production wells. In some embodiments, a hydrocarbon-bearing reservoir is a depleted oil reservoir. In some embodiments, a hydrocarbon-bearing reservoir is a depleted gas reservoir. In some embodiments, a hydrocarbon-bearing reservoir will contain a hydrocarbon-bearing fluid, such as crude oil, natural gas, petroleum, diesel fuel, gasoline, or any other fluids that include an amount of hydrocarbons.

In some embodiments, the aqueous brine comprises a concentration of inorganic salts dissolved in water. The aqueous brine may include naturally-occurring brines (for example, seawater), synthetic brines, or both. In some embodiments, the aqueous brine comprises deionized water. In some embodiments, the aqueous brine comprises one or more alkali or alkaline earth metal halides. Representative examples of suitable alkali or alkaline earth metal halides include calcium chloride, calcium bromide, sodium chloride, sodium bromide, magnesium chloride, magnesium bromide and combinations thereof.

In a non-limiting illustrative embodiment, an aqueous brine can have a salinity sufficient to prevent or inhibit one or more of methanogenesis and sulphate-reduction activity in the hydrocarbon-bearing reservoir, i.e., prevent or inhibit the generation of CH₄ and/or H₂S. In some embodiments, an aqueous brine can have a salinity of greater than or equal to about 200 ppt. In some embodiments, an aqueous brine can have a salinity of greater than or equal to about 210 ppt. In some embodiments, an aqueous brine can have a salinity of greater than or equal to about 215 ppt. In some embodiments, an aqueous brine can have a salinity of greater than or equal to about 225 ppt. In some embodiments, an aqueous brine can have a salinity of greater than or equal to about 230 ppt. In some embodiments, an aqueous brine can have a salinity of greater than or equal to about 235 ppt. In some embodiments, an aqueous brine can have a salinity of greater than or equal to about 240 ppt. In some embodiments, an aqueous brine can have a salinity of greater than or equal to about 245 ppt. In some embodiments, an aqueous brine can have a salinity of greater than or equal to about 250 ppt. In some embodiments, an aqueous brine can have a salinity of no more than about 350 ppt. In some embodiments, an aqueous brine can have a salinity of no more than about 325 ppt. In some embodiments, an aqueous brine can have a salinity of no more than about 300 ppt. The salinity level of a hydrocarbon-bearing reservoir can be determined by methods known in the art such as, for example, salinity from density measurement (specific gravity), full water ionic analysis (cations and anions), gravimetric determination of dissolved solids, etc.

Following the identification of a hydrocarbon-bearing reservoir containing a hydrocarbon fluid and a brine having a sufficient salinity level to prevent or inhibit one or more of methanogenesis and sulphate-reduction activity in the hydrocarbon-bearing reservoir, a concentration of hydrogen in a sample collected from the hydrocarbon-bearing reservoir is determined. This sampling and analysis will typically be anaerobic. Methods and equipment for collection of gases from a formation are well known to the art. See, e.g., WO 02/34931. In some embodiments, the sample collected is a liquid sample. In some embodiments, the sample collected is a hydrocarbon fluid sample. In some embodiments, the sample collected is a water sample. As one skilled in the art will appreciate, the concentration of hydrogen in the sample is an indicator of whether the microbial production of hydrogen through the anaerobic microbial fermentation of native hydrogen producing microorganisms to produce hydrogen in-situ from the hydrocarbon fluid in the hydrocarbon-bearing reservoir is an amount sufficient to collect the generated hydrogen for further processing and utilization in, for example, transportation, electric, natural gas, or as a feedstock. In some embodiments, the concentration of hydrogen in the sample can be determined by techniques well known in the art, for example, gas chromatography with thermal conductivity detection (GC-TCD).

In some embodiments, a concentration of hydrogen in the sample below 1000 parts per million (ppm) or below about 900 ppm, or below about 800 ppm, or below about 700 ppm, or below about 600 ppm, or below about 500 ppm, or below about 400 ppm, or below about 300 ppm, or below about 200 ppm, or below about 100 ppm, or below about 50 ppm, or below about 10 ppm is an indicator that the microbial production of hydrogen through the anaerobic microbial fermentation of native hydrogen producing microorganisms to produce hydrogen in-situ from the hydrocarbon fluid in the hydrocarbon-bearing reservoir is an amount insufficient to collect the hydrogen for further processing and utilization.

Accordingly, in order to generate additional hydrogen in the hydrocarbon-bearing reservoir, at least one non-native or indigenous hydrogen producing microorganism or microbial nutrients are introduced into hydrocarbon-bearing reservoir to diversify the microbiological abundance of the native hydrogen-producing microorganisms in the hydrocarbon-bearing reservoir to produce hydrogen in-situ from the hydrocarbon fluid in the hydrocarbon-bearing reservoir through anaerobic microbial fermentation. In some embodiments, that at least one non-native or indigenous hydrogen producing microorganism or microbial nutrients are introduced into hydrocarbon-bearing reservoir to produce a concentration of hydrogen of from about 1 ppm to about 1000 ppm. In some embodiments, that at least one non-native or indigenous hydrogen producing microorganism or microbial nutrients are introduced into hydrocarbon-bearing reservoir to produce a concentration of hydrogen of from about 100 ppm to about 900 ppm. In some embodiments, the concentration of hydrogen in the sample is zero.

In some embodiments, suitable non-native or indigenous hydrogen producing microorganisms include, for example, a microorganism not naturally present in the hydrocarbon fluid; a strain of microorganisms not naturally present in the hydrocarbon fluid; a species of microorganisms not naturally present in the hydrocarbon fluid; a genus of microorganisms not naturally present in the hydrocarbon fluid; and/or a microorganism naturally present in the hydrocarbon fluid.

In some embodiments, a non-native or indigenous hydrogen producing microorganism may have a genus of one or more of Syntrophobacter, Syntrophus, Syntrophomonas, Thermoanaerobacter, Thermotoga, Pseudothermotoga, Thermoanaerobacterium, Fervidobacterium, Thermosipho, Haloanaerobium, Acetoanaerobium, Anaerobaculum, Geotoga, Petrotoga, Thermococcus, Pyrococcus, Clostridium, Enterobacter, Klebsiella, Ethanoligenens, Pantoea, Escherichia, Bacillus, Caldicellulosiruptor, Pelobacter, Caldanaerobacter, Marinitoga, Oceanotoga, Defluviitoga, Kosmotoga, Caloranaerobacter, Marinobacter, and Tolumonas. In some embodiments, the method herein is targeting halophilic fermentation. In some embodiments, Haloanaerobium is the target organism.

In some embodiments, a non-native or indigenous hydrogen producing microorganism may include a genus of Halomonas. In some embodiments, a non-native or indigenous hydrogen producing microorganism may be a Halomonas sp. G11 strain.

In some embodiments, as may be combined with the preceding paragraphs, a non-native or indigenous hydrogen producing microorganism may be introduced into the hydrocarbon-bearing reservoir and accompanied during, after or upon its introduction by at least one nutrient selected to promote the growth of native or non-native or indigenous hydrogen producing microorganisms. The nutrients are selected preferentially to promote the growth of the non-native or indigenous hydrogen producing microorganism in preference to at least one, to at least some or to all of the native hydrogen producing microorganisms in the deposit. Suitable nutrients include, for example, one or more of one or more salts selected from phosphates, and/or halides, and/or nitrates, ammonium salts, and nitrogenous salts; and/or one or more carbohydrates selected from sugars, and/or starches; and/or one or more vitamins; and/or complex nutrients, optionally comprising yeast extracts, corn steep liquor, biomass, bacterial an/or algal biomass.

In some embodiments, as may be combined with the preceding paragraphs, a non-native or indigenous hydrogen producing microorganism may be introduced into the hydrocarbon-bearing reservoir and accompanied during, after or upon its introduction by at least one pH regulator selected to regulate the pH environment in which the microorganism resides in the hydrocarbon-bearing reservoir. The pH regulator may be selected to regulate the pH of the hydrogen producing microorganism environment in the deposit to a pH within the range of from about 5 to about 9, or from about 6 to about 8 and/or from about 6 to about 7. In some embodiments, the pH regulator may optionally also serve as a nutrient. For example, a phosphate can acts as both a nutrient and as a buffering agent.

In some embodiments, a concentration of hydrogen in the sample equal to or greater than 1000 ppm is an indicator that the microbial production of hydrogen through the anaerobic microbial fermentation of native hydrogen producing microorganisms to produce hydrogen in-situ from the hydrocarbon fluid in the hydrocarbon-bearing reservoir is an amount sufficient to collect the hydrogen for further processing and utilization. Accordingly, in some embodiments, at least a portion of a hydrogen-rich material comprising hydrogen is collected from the hydrocarbon-bearing reservoir. The hydrogen-rich material can be sent for further processing and utilization in, for example, transportation, electric, natural gas, or as a feedstock.

The following examples are provided to further illustrate the present process and its benefits. The examples are meant to be illustrative and not limiting.

### EXAMPLES

Two high salinity produced water samples were collected from oilfield water treatment facilities. The two samples were determined to contain no hydrogen. The salinity of the two samples were determined from complete water ionic analysis and are reported in Table 1.

TABLE 1

| Parameter | Sample A | Sample B |
|---|---|---|
| Salinity, ppt | 133 | 117 |

### Test Protocol

Bacterial seed cultures (seed cultures A and B) were grown from indigenous bacteria present in the two produced water samples. In this case, no external bacteria were introduced to the culture. The seed cultures were grown by incubation at 30° C. in produced water plus additional nutrients for 4 weeks. An adenosine triphosphate (ATP) extraction and quantification was conducted on the seed cultures to confirm a microbial load of greater than 10\hat{ }5 ME/ml in the culture after initial incubation and growth.

Two sets of duplicate serum bottles (serum bottles 1 to 4, see Table 2) were prepared containing a sterilized produced water sample with modified salinity, a subsample of the corresponding seed culture, acid washed sand, and supplementary nutrients (bacterial media 3 formulation). The salinity of media 3 was adjusted to 200 ppt. The bottles were deoxygenated by sparging with high purity nitrogen and then crimp sealed retaining a nitrogen headspace.

The serum bottles were incubated at 30° C. for 3 months, followed by collection and analysis of a head space gas sample from each serum bottle. Natural gas analysis by GC-MS (gas chromatography-mass spectroscopy) for hydrocarbons to C6+, CO₂, H₂, H₂S, N₂, and O₂+Ar was conducted on each headspace gas sample.

### Microbial Media 3

50:50 produced water: synthetic produced water mixtures with addition of potassium phosphate monobasic, sodium, acetate, sodium propionate, sodium butyrate, sodium lactate, and sodium formate were prepared as follows:

0.06 ppt Potassium Phosphate Monobasic
1.20 ppt Sodium Acetate
0.12 ppt Sodium Propionate
0.11 ppt Sodium Butyrate
0.40 ppt Sodium lactate
0.60 ppt Sodium Formate

The salinity was adjusted to target salinity of 200 ppt with sodium chloride. There was no iron source addition in this media. The media is autoclaved at 120° C. for 1 hour to sterilize.

### Test Matrix

The test matrix is set forth below in Table 2.

TABLE 2

| | | Produced | | | |
|---|---|---|---|---|---|
| Serum | | Water | Media | Seed | |
| Bottle | Substrate | Sample | Formulation | Culture | Salinity |
| #1 | 40 g acid | Sample A | Media 3 | Seed | 200 ppt |
| | washed sand | | | culture A | |
| #2 | 40 g acid | Sample A | Media 3 | Seed | 200 ppt |
| | washed sand | | | culture A | |
| #3 | 40 g acid | Sample B | Media 3 | Seed | 200 ppt |
| | washed sand | | | culture B | |
| #4 | 40 g acid | Sample B | Media 3 | Seed | 200 ppt |
| | washed sand | | | culture B | |

### Results

The high salinity growth tests with produced water and microbial cultures demonstrated that microbial metabolism is dependent on both brine salinity and microbial consortium. The results are set forth below in Table 3.

At >=200 ppt salinity the activity of fermentative bacteria resulted in the accumulation of up to 950 ppm (0.095 Mol %) H₂ in headspace of test bottles #1 and #2 over a period of 3 months. Sulphate reducing bacteria (SRB) activity was precluded due to the high salinity in sample A and the microbial community in seed culture A.

At <=200 ppt salinity halophilic SRB generated high levels of H₂S in the test bottles #3 and #4. Fermentative bacteria activity generated hydrogen which is consumed as an electron donor to support SRB activity, based on the microbial community in seed culture B.

TABLE 3

| | Bottle #1 | Bottle #2 | Bottle #3 | Bottle #4 |
|---|---|---|---|---|
| | Water | Water | Water | Water |
| | Sample A | Sample A | Sample B | Sample B |
| | 200 ppt | 200 ppt | 200 ppt | 200 ppt |
| Description | Salinity | Salinity | Salinity | Salinity |
| Components | Mole % | Mole % | Mole % | Mole % |
| Hydrogen | 0.082 | 0.095 | <0.005 | <0.005 |
| CO₂ | 0.950 | 6.006 | 2.637 | 2.757 |
| Ethane | <0.001 | <0.001 | <0.001 | <0.001 |
| Oxygen + Argon | <0.005 | 0.591 | <0.005 | <0.005 |
| Nitrogen | 98.967 | 93.306 | 96.089 | 95.971 |
| H₂S | <0.05 | <0.05 | 1.272 | 1.271 |
| Methane | <0.001 | <0.001 | <0.001 | <0.001 |
| Propane | <0.001 | <0.001 | <0.001 | <0.001 |
| Isobutane | <0.001 | <0.001 | <0.001 | <0.001 |
| Normal Butane | <0.001 | <0.001 | <0.001 | <0.001 |
| Isopentane | <0.001 | <0.001 | <0.001 | <0.001 |
| Normal Pentane | <0.001 | <0.001 | <0.001 | <0.001 |
| C6+ | 0.001 | 0.001 | 0.002 | 0.001 |
| Total | 100.000 | 100.000 | 100.000 | 100.000 |

According to an aspect of the disclosure, a process comprises:
identifying a hydrocarbon-bearing reservoir containing a hydrocarbon fluid and a brine having a sufficient salinity level to prevent or inhibit one or more of methanogenesis and sulphate-reduction activity, and
responsive to a concentration of hydrogen being below 1000 parts per million in a sample collected from the hydrocarbon-bearing reservoir, introducing at least one non-native or indigenous hydrogen producing microorganism or stimulating the activity of at least one indigenous bacteria capable of stimulating a microbial consortium to increase a production rate of hydrogen in the hydrocarbon-bearing reservoir.

In one or more additional illustrative embodiments, as may be combined with the preceding paragraphs, the brine has a salinity greater than or equal to about 200 parts per thousand (ppt).

In one or more additional illustrative embodiments, as may be combined with the preceding paragraphs, the brine has a salinity greater than or equal to about 210 ppt.

In one or more additional illustrative embodiments, as may be combined with the preceding paragraphs, the brine has a salinity greater than or equal to about 200 ppt and up to about 350 ppt.

In one or more additional illustrative embodiments, as may be combined with the preceding paragraphs, the brine has a salinity greater than or equal to about 200 ppt and up to about 300 ppt.

In one or more additional illustrative embodiments, as may be combined with the preceding paragraphs, the brine has a salinity greater than or equal to about 200 ppt and up to about 350 ppt.

In one or more additional illustrative embodiments, as may be combined with the preceding paragraphs, the hydrocarbon-bearing reservoir is a depleted oil reservoir.

In one or more additional illustrative embodiments, as may be combined with the preceding paragraphs, the hydrocarbon-bearing reservoir is a depleted gas reservoir.

In one or more additional illustrative embodiments, as may be combined with the preceding paragraphs, the hydrocarbon fluid is an oil.

In one or more additional illustrative embodiments, as may be combined with the preceding paragraphs, the hydrocarbon fluid is natural gas.

In one or more additional illustrative embodiments, as may be combined with the preceding paragraphs, the at least one non-native or indigenous hydrogen producing microorganism comprises a genus of one or more of Syntrophobacter, Syntrophus, Syntrophomonas, Thermoanaerobacter, Thermotoga, Pseudothermotoga, Thermoanaerobacterium, Fervidobacterium, Thermosipho, Haloanaerobium, Acetoanaerobium, Anaerobaculum, Geotoga, Petrotoga, Thermococcus, Pyrococcus, Clostridium, Enterobacter, Klebsiella, Ethanoligenens, Pantoea, Escherichia, Bacillus, Caldicellulosiruptor, Pelobacter, Caldanaerobacter, Marinitoga, Oceanotoga, Defluviitoga, Kosmotoga, Marinobacter, and Tolumonas.

In one or more additional illustrative embodiments, as may be combined with the preceding paragraphs, the at least one non-native or indigenous hydrogen producing microorganism comprises a genus of Halomonas.

In one or more additional illustrative embodiments, as may be combined with the preceding paragraphs, the at least one non-native or indigenous hydrogen producing microorganism comprises Halomonas sp. G11 strain.

In one or more additional illustrative embodiments, as may be combined with the preceding paragraphs, the process further comprises introducing at least one nutrient selected to promote the growth of the at least one non-native or indigenous hydrogen producing microorganism.

In one or more additional illustrative embodiments, as may be combined with the preceding paragraphs, the at least one nutrient comprises one or more of one or more salts selected from the group consisting of a phosphate salt, a halide salt, a nitrate salt, an ammonium salt and a nitrogenous salt; one or more carbohydrates selected from the group consisting of a sugar, and a starch; one or more vitamins and a complex nutrient.

In one or more additional illustrative embodiments, as may be combined with the preceding paragraphs, the process further comprises introducing at least one pH regulator selected to regulate the pH environment in which the at least one non-native or indigenous hydrogen producing microorganism resides in the hydrocarbon-bearing reservoir.

In one or more additional illustrative embodiments, as may be combined with the preceding paragraphs, the at least one pH regulator regulates the pH of the hydrocarbon-bearing reservoir within the range of from about 5 to about 9.

According to another aspect of the disclosure, a process comprises:
identifying a hydrocarbon-bearing reservoir containing a hydrocarbon fluid and a brine having a sufficient salinity level to prevent or inhibit one or more of methanogenesis and sulphate-reduction activity, and
responsive to a concentration of hydrogen being equal to or greater than 1000 parts per million in a sample collected from the hydrocarbon-bearing reservoir, collecting at least a portion of a hydrogen-rich material comprising hydrogen from the hydrocarbon-bearing reservoir.

In one or more additional illustrative embodiments, as may be combined with the preceding paragraphs, the brine has a salinity greater than or equal to about 200 ppt.

In one or more additional illustrative embodiments, as may be combined with the preceding paragraphs, the brine has a salinity greater than or equal to about 210 ppt.

In one or more additional illustrative embodiments, as may be combined with the preceding paragraphs, the brine has a salinity greater than or equal to about 200 ppt and up to about 350 ppt.

In one or more additional illustrative embodiments, as may be combined with the preceding paragraphs, the brine has a salinity greater than or equal to about 200 ppt and up to about 300 ppt.

In one or more additional illustrative embodiments, as may be combined with the preceding paragraphs, the brine has a salinity greater than or equal to about 200 ppt and up to about 350 ppt.

In one or more additional illustrative embodiments, as may be combined with the preceding paragraphs, the hydrocarbon-bearing reservoir is a depleted oil reservoir.

In one or more additional illustrative embodiments, as may be combined with the preceding paragraphs, the hydrocarbon-bearing reservoir is a depleted gas reservoir.

In one or more additional illustrative embodiments, as may be combined with the preceding paragraphs, the hydrocarbon fluid is an oil.

In one or more additional illustrative embodiments, as may be combined with the preceding paragraphs, the hydrocarbon fluid is natural gas.

Various features disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present disclosure and are disclosed herein just as if each and every such sub-combination was individually and explicitly disclosed herein.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

What is claimed is:

1. A process, comprising:
identifying a hydrocarbon-bearing reservoir containing a hydrocarbon fluid and a brine having a sufficient salinity level to prevent or inhibit one or more of methanogenesis and sulphate-reduction activity; and
responsive to a concentration of hydrogen being below 1000 parts per million in a sample collected from the hydrocarbon-bearing reservoir, introducing at least one non-native or indigenous hydrogen producing microorganism or stimulating the activity of at least one indigenous bacteria capable of stimulating a microbial consortium to increase a production rate of hydrogen in the hydrocarbon-bearing reservoir.

2. The process according to claim 1, wherein the brine has a salinity greater than or equal to about 200 parts per thousand (ppt).

3. The process according to claim 1, wherein the brine has a salinity greater than or equal to about 200 ppt and up to about 350 ppt.

4. The process according to claim 1, wherein the hydrocarbon-bearing reservoir is a depleted oil reservoir.

5. The process according to claim 1, wherein the hydrocarbon-bearing reservoir is a depleted gas reservoir.

6. The process according to claim 1, wherein the hydrocarbon fluid is an oil.

7. The process according to claim 1, wherein the hydrocarbon fluid is natural gas.

8. The process according to claim 1, wherein the at least one non-native or indigenous hydrogen producing microorganism comprises a genus of one or more of Syntrophobacter, Syntrophus, Syntrophomonas, Thermoanaerobacter, Thermotoga, Pseudothermotoga, Thermoanaerobacterium, Fervidobacterium, Thermosipho, Haloanaerobium, Acetoanaerobium, Anaerobaculum, Geotoga, Petrotoga, Thermococcus, Pyrococcus, Clostridium, Enterobacter, Klebsiella, Ethanoligenens, Pantoea, Escherichia, Bacillus, Caldicellulosiruptor, Pelobacter, Caldanaerobacter, Marinitoga, Oceanotoga, Defluviitoga, Kosmotoga, Marinobacter, and Tolumonas.

9. The process according to claim 1, wherein the at least one non-native or indigenous hydrogen producing microorganism comprises a genus of Halomonas.

10. The process according to claim 1, further comprising introducing at least one nutrient selected to promote the growth of the at least one non-native or indigenous hydrogen producing microorganism.

11. The process according to claim 10, wherein the at least one nutrient comprises one or more of one or more salts selected from the group consisting of a phosphate salt, a halide salt, a nitrate salt, an ammonium salt and a nitrogenous salt; one or more carbohydrates selected from the group consisting of a sugar, and a starch; one or more vitamins and a complex nutrient.

12. The process according to claim 1, further comprising introducing at least one pH regulator selected to regulate the pH environment in which the at least one non-native or indigenous hydrogen producing microorganism resides in the hydrocarbon-bearing reservoir.

13. The process according to claim 12, where in the at least one pH regulator regulates the pH of the hydrocarbon-bearing reservoir within a range of from about 5 to about 9.

14. A process, comprising:
identifying a hydrocarbon-bearing reservoir containing a hydrocarbon fluid and a brine having a sufficient salinity level to prevent or inhibit one or more of methanogenesis and sulphate-reduction activity; and
responsive to a concentration of hydrogen being equal to or greater than 1000 parts per million in a sample collected from the hydrocarbon-bearing reservoir, collecting at least a portion of a hydrogen-rich material comprising hydrogen from the hydrocarbon-bearing reservoir.

15. The process according to claim 14, wherein the brine has a salinity greater than or equal to about 200 ppt.

16. The process according to claim 14, wherein the brine has a salinity greater than or equal to about 200 ppt and up to about 350 ppt.

17. The process according to claim 14, wherein the hydrocarbon-bearing reservoir is a depleted oil reservoir.

18. The process according to claim 14, wherein the hydrocarbon-bearing reservoir is a depleted gas reservoir.

19. The process according to claim 14, wherein the hydrocarbon fluid is an oil.

20. The process according to claim 14, wherein the hydrocarbon fluid is natural gas.
